(19) United States
(12) Patent Application Publication
(10) Pub. No.: US 2006/0292094 A1
(43) Pub. Date: Dec. 28, 2006

(54) Composition and method of protection against UV irradiation

(76) Inventor: Robert Bell, Miami, FL (US)

Correspondence Address: AMIN LAW, LLC, 217 N. JEFFERSON ST., SUITE 100, CHICAGO, IL, 60661, US

(21) Appl. No.: 11/165,686

(22) Filed: Jun. 24, 2005

(51) Int. Cl. A61K 36/82; A61K 31/59; A61K 31/355; A61K 8/27; A61K 33/04; A61K 36/886

(52) U.S. Cl. 424/59; 424/729; 424/725; 424/773; 424/641; 424/702; 514/167; 514/458; 424/60; 424/744

(57) ABSTRACT

The present invention relates to a composition for use in a nutritional product, dietary supplement, or pharmaceutical compound wherein such composition is used to protect the skin against the effects of ultraviolet (UV) irradiation from the sun or other sources, including but not limited to sunburn, skin redness, swelling, immune suppression, photo-aging, photodamage, and skin cancer. The invention relates to a composition containing a variety of antioxidants, vitamins, and herbal ingredients. The present invention further relates to a method of administering a therapeutically effective amount of the composition ingredients to protect the skin against the effects of UV irradiation.

### FIELD OF INVENTION

The present invention relates to a composition for use in a nutritional product, dietary supplement, or pharmaceutical compound wherein such composition is used to protect the skin against the effects of ultraviolet (UV) irradiation from the sun or other sources, including but not limited to sunburn, skin redness, swelling, immune suppression, photo-aging, photodamage, and skin cancer. The present invention further relates to a method of administering a therapeutically effective amount of the composition ingredients to protect the skin against the effects of UV irradiation.

### BACKGROUND OF THE INVENTION

UV irradiation is an environmental factor which greatly impacts and affects the human skin. The skin is made up of the epidermal layer which is the barrier to outside environmental factors and includes the stratum corneum; the dermal layer which is the underlying layer comprised of collagen and elastin; and the subcutaneous layer. UV irradiation can produce beneficial effects on the skin, such as the production of Vitamin D, or harmful effects on the skin, such as sunburn, photo-aging, and skin cancer. Sources of UV radiation include the sun, and artificial sources such as tanning beds, and sun lamps. UV irradiation which is damaging to the skin includes UV-B radiation, which ranges from 290 to 320 nm, and UV-A radiation, which ranges from 320 to 400 nm.

The beneficial effects to the skin of UV radiation exposure include the production of Vitamin D which is necessary for the body to maintain calcium levels, good muscle health, and healthy bone formation. Vitamin D can also be derived from diet.

The harmful effects to the skin of UV radiation exposure include sunburn, skin redness, pigmentation changes, swelling, immune suppression, photo-aging, photodamage, and skin cancer. The earth's ozone layer blocks and screens UV radiation from the sun; however, due to the decrease in the ozone layer, the harmful effects of UV radiation exposure are increasing and worsening. Sunburn, for example, can range from mild to severe depending upon the skin type and the amount of UV radiation exposure and can produce tenderness, pain, swelling, blistering, erythema, and cell damage in the epidermal layer, including the stratum corneum. Photo-aging and premature skin aging results from exposure to UV radiation, specifically UV-B radiation, and can cause the skin to thicken, lose its elasticity, wrinkle, and change pigments. Skin cancer or photocarcinogenesis is the most damaging effect of UV irradiation and the most common form of cancer in the United States. This type of photodamage relates to the DNA of the skin being damaged as a result of UV irradiation and the skin's natural repair mechanisms being unable to repair the resulting DNA mutations. In the United States, it is estimated that over 100,000 people will be diagnosed in 2005 with melanoma, a serious form of skin cancer which generally results from intermittent, but very intense, UV radiation exposure and that close to 8,000 people will die from skin cancer.

The skin defends itself from damaging environmental factors, including UV irradiation, through its immune function, antioxidant functions, and responses to skin inflammation. UV irradiation causes the production of free radicals which can damage the skin's DNA, proteins and other essential components. The epidermal layer of the skin includes antioxidants such as Vitamin C, carotenoids, and Vitamin E, which remove and defend against these damaging free radicals produced from UV irradiation. The epidermal layer of skin also protects the skin from damaging effects by performing epidermal hyperplasia which is the thickening of the skin to increase its tolerance to environmental factors, including UV radiation exposure.

Many protections of the skin against the effects of UV irradiation exist. Sunscreens are one form of skin protection against the effects of UV irradiation. The protection afforded by sunscreens is measured by the sun protection factor (SPF) which is the amount of UV irradiation that will cause minimally perceptible skin redness on skin protected by an application of 2 mg/cm² of sunscreen divided by the amount of UV irradiation that will cause skin redness on skin not protected by sunscreen. For example, a sunscreen designated as SPF 15 does not afford as much protection from UV irradiation as a sunscreen designated as SPF 30. However the actual SPF afforded by a sunscreen can be affected by other factors, including skin type, water exposure, and sweating. Sunscreens exist which are comprised of active ingredients, including but not limited to para-aminobenzoic acid derivatives (PABA) which can stain clothing due to the use of alcohol and alone do not provide the desired SPF alone; anthranilates which do not provide adequate UV-B or UV-A I protection; benzophenones which only provide UV-B and UV-A II protection; octyl methoxycinnamate; avobenzone which protects against UV-A I and UV-A II and supplements UV-B protectors; and titanium dioxide and zinc oxide which do not cause skin irritation and protect well against UV-B and UV-A I irradiation. A problem with traditional sunscreens is that they alone do not provide adequate protection from the effects of UV irradiation and protection against all ranges of UV-B and UV-A irradiation. Therefore, a sunscreen is needed which provides adequate skin protection against all ranges of UV-B and UV-A irradiation alone or in combination with other sunscreens and skin protection.

Another currently used skin protection against the effects of UV irradiation is the use of a variety of antioxidants, including Vitamin C, Vitamin E, beta-carotene and alpha-lipoic acid. Vitamin C reduces inflammation from sunburn, protects the DNA in skin cells from photodamage, prevents immunosuppression, and promotes the production of collagen in the skin. Beta-carotene supplementation has also been utilized to support skin health, promote the production of melanin, and support the body's natural defenses against photodamage from UV radiation exposure by maintaining normal immune response and scavenging free radicals resulting from UV radiation exposure. Vitamin E also supports healthy response to UV irradiation. A therapeutically effective amount of antioxidants alone or in combination with other ingredients is needed for the protection of the skin against the effects of UV irradiation.

Currently topical and oral methods of administering protection of the skin against the effects of UV irradiation exist including lotions, creams, cosmetics and patches. For example, although not specifically for the effects of UV irradiation, Polypodium leucotomos has been administered orally and topically to systemically provide increased protection against skin redness and to treat inflammatory diseases, which are several of the effects of UV irradiation. Beta-carotene oral supplementation has been used to provide UV irradiation protection. Topical application of sunscreen and antioxidants is the most common form of administering skin protection against UV irradiation; however, the effectiveness of these methods varies depending upon skin type, exposure to water, and sweating. Additionally, oral administration of protection of the skin against UV irradiation has not been therapeutically effective. Therefore, a method of oral administration of a therapeutically effective amount of a composition for the protection of the skin against the effects of UV irradiation is needed wherein the effectiveness is not affected by skin type, exposure to water, and sweating.

Another method commonly used to protect the skin against the effects of UV irradiation relates to the wearing of protective clothing including hats, long sleeve shirts, pants, socks, and sunglasses. However, for certain activities, such as running and biking during hot weather, wearing protective clothes is not comfortable, feasible or is difficult. A method of administering protection of the skin against the effects of UV irradiation is needed wherein protection is still afforded to the skin without covering the entire body.

Currently the use of a topical sunscreen having an SPF of 8 or greater is commonly used to protect the skin against the effects of UV irradiation. However, the use of a topical sunscreen having an SPF greater than SPF 8 blocks the body's natural production of Vitamin D. Regular Vitamin D intake is necessary because Vitamin D helps to improve bone and muscle health. Generally, Vitamin D is derived from diet and from UV radiation exposure. If the body's production of Vitamin D is deficient then Vitamin D levels can most safely be increased through dietary supplements or fortified foods. Therefore, a composition is needed which provides SPF protection against UV radiation exposure and also supplements the body's Vitamin D intake.

Additionally, compositions exist for the promotion of healthy skin which contain at least lycopene, biotin, selenium, chromium and carotenoids in combination with other vitamins and minerals. Other sunscreen compositions exist which only include a cyanin, preferably an anthocyanin and a betacyanin. Compositions also exist which contain fruit extracts preferably from pomegranate, hydrophobic and hydrophilic moisturizing agents, manganese ascorbate, mono- or poly-hydroxy or tannic acid, and carriers for the treatment of dermatological conditions. The previously mentioned composition has further embodiments containing a sunscreen component, an anti-inflammatory component, and an antioxidant component. Topical compositions also exist for the reduction of UV radiation effects including reduced L-glutathione, selenium, and epidermal growth factor. Sports drinks also exist consisting of carotenoids, water, and a variety of antioxidants for the protection against the harmful effects of solar radiation. Methods of administering compositions of antioxidants also exist for protecting skin from the effects of UV rays. However, there is a need for a composition which is orally administered in a therapeutically effective combination of a variety of antioxidants, vitamins, immuno-modulatory agents and herbal ingredients for the protection of skin against the effects of UV irradiation.

A composition for use in a nutritional product, dietary supplement, or pharmaceutical compound wherein such composition is used to protect the skin against the effects of UV irradiation is needed which provides adequate protection alone or in combination with other sunscreens; protection against all ranges of UV-B and UV-A irradiation; a therapeutically effective amount of antioxidants alone or in combination with other ingredients; a method of effective protection that is not affected by skin type, exposure to water, and sweating; a method of protection wherein clothing covering the entire body is not needed; and SPF protection that also supplements the body's Vitamin D intake. There is also a need for a method of administering a therapeutically effective amount of the composition ingredients for the protection of the skin against the effects of UV irradiation, including but not limited to sunburn, skin redness, swelling, immune suppression, photo-aging, photodamage, and skin cancer.

### SUMMARY OF THE INVENTION

The present invention relates to a composition for use in a nutritional product, dietary supplement, or pharmaceutical compound wherein such composition is used for the protection of the skin against the effects of UV irradiation from the sun or other sources, including but not limited to sunburn, skin redness, swelling, immune suppression, photo-aging, photodamage, and skin cancer. The present invention further relates to a method of administering a therapeutically effective amount of the composition ingredients for the protection of the skin against the effects of UV irradiation.

A general object of the present invention is to support the skin's natural defenses against the sun.

Another general object of the present invention is to protect skin against UV radiation from exposure and damage, including but not limited to the prevention of photo-aging, photodamage, and some forms of skin cancer.

A further object of the present invention is to promote overall healthy skin function and to promote healthier, youthful skin.

More particularized objects of the present invention are to support skin immune function, support skin antioxidant function, and to maintain healthy response to skin inflammation.

A further object of the present invention is to effectively supplement SPF obtained from topical UV irradiation protection.

Another particularized object of the present invention is to supplement the body's Vitamin D intake.

The present invention relates to a therapeutically effective combination of a variety of antioxidants, vitamins, immuno-modulatory agents and herbal ingredients for the protection of skin against the effects of UV irradiation. The combination of ingredients of the present invention include, but are not limited to beta-carotene, Vitamin E, Vitamin C, Pycnogenol®, selenium, zinc, Vitamin D, para-aminobenzoic acid, and a variety of other antioxidants, immuno-modulatory agents, and herbal ingredients.

There is a need for a therapeutically effective combination of ingredients to adequately protect the skin against all ranges of UV irradiation and the effects of UV irradiation for use alone or in combination with other sunscreens. One preferred embodiment of the present invention relates to a therapeutically effective combination of ingredients for the protection of skin against the effects of UV irradiation to be used alone or in combination with other sunscreens and to be administered orally. The prior art has failed to provide protection for the skin from the effects of UV irradiation which provides adequate protection alone or in combination with other sunscreens; protection against all ranges of UV-B and UV-A irradiation; a therapeutically effective amount of antioxidants alone or in combination with other ingredients; a method of effective protection that is not affected by skin type, exposure to water, and sweating; a method of protection wherein clothing covering the entire body is not needed; and SPF protection that also supplements the body's Vitamin D intake. The prior art has also failed to provide a method of administering a therapeutically effective amount of the composition ingredients for the protection of the skin against the effects of UV irradiation, including, but not limited to sunburn, skin redness, swelling, immune suppression, photo-aging, photodamage, and skin cancer.

### DETAILED DESCRIPTION OF THE INVENTION

The present invention relates to a composition for use in a nutritional product, dietary supplement, or pharmaceutical compound wherein such composition is used for the protection of the skin against the effects of UV irradiation from the sun or other sources. The present invention further relates to a method of administering a therapeutically effective amount of the composition ingredients for the protection of the skin against the effects of UV irradiation.

The composition includes, without limitation, at least one antioxidant component, at least one vitamin component, at least one immuno-modulatory agent and at least one herbal component in a therapeutically effective amount for the adequate protection of the skin against UV irradiation from the sun or artificial sources and its effects.

The at least one antioxidant component of the present invention preferably includes at least one carotenoid, at least one polyphenol-containing compound, ellagic acid, at least one betacyanin, selenium, and zinc.

In one preferred embodiment, the at least one carotenoid includes beta-carotene obtained from D-salina algae combined with other natural carotenoids, such as, but not limited to alpha-carotene, cryptoxanthin, zeaxanthin, or lutein. Carotenoids are antioxidants contained in a variety of plants, fruits and vegetables having yellow, orange, and red pigments. Certain carotenoids, including beta-carotene, are converted by the body to Vitamin A which helps to maintain healthy skin, healthy skin immune function, and general cellular health. UV irradiation can deplete carotenoid levels found in the body which decreases the skin's natural defense against damaging free radicals produced from UV irradiation. Carotenoids, among other functions, support skin health, prevent UV irradiation-induced skin redness, promote the production of melanin which provides brown coloration in varying degrees to the skin surface, and support the body's natural defenses against photodamage from UV irradiation by maintaining normal skin immune response and scavenging free radicals resulting from UV radiation exposure. Prolonged beta-carotene supplementation has been shown to increase the yellow pigmentation of the skin to provide protection from UV irradiation and to promote the production of melanin. In one embodiment, the at least one carotenoid includes at least one of the following: synthetic beta-carotene, lutein, lycopene, astaxanthin, cryptoxanthin, zeaxanthin, or similarly therapeutic combinations thereof. In another embodiment, the at least one carotenoid includes a synthetic carotenoid, such as cantaxanthin or other similarly therapeutic ingredient.

In one preferred embodiment, the at least one polyphenol-containing compound includes pine bark extract or other similar extract. In another preferred embodiment the polyphenol-containing compound includes Pycnogenol® which is a combination of proanthocyanidins, catechins, and phenolic acids. Pycnogenol® increases the amount of time it takes for unprotected skin to develop sunburn, supports the skin structure, and prevents the degradation of collagen and elastin contained in the epidermal layer of the skin by protecting and recycling Vitamin C that manufactures collagen and binding to and inhibiting collagen degrading enzymes.

In another preferred embodiment, the at least one polyphenol-containing compound includes green tea extract or other similar extract. The green tea extract includes catechins and polyphenols and promotes healthy immune function, reduces skin sensitivity to sun damage, prevents skin inflammation, and specifically protects the skin against UV-B radiation exposure. In this preferred embodiment, the at least one antioxidant component is obtained from white tea, coffee, chocolate, grape skins, grape seeds, apple extract, pine bark, onions, berries, or other similar herbs and fruits.

In one preferred embodiment, the ellagic acid is obtained from pomegranate extract. Pomegranate extract is plant-based and contains anthocyanins and hydrolysable tannins which are strong antioxidants and anti-inflammatory agents. Pomegranate extract increases SPF with short-term supplementation and reduces skin cell damage from UV irradiation exposure, specifically UV-B radiation. In this preferred embodiment, the ellagic acid is obtained from raspberry seed, strawberry seed, guava, date palm or other similar sources of ellagic acid.

The at least one antioxidant component of the present invention preferably includes at least one betacyanin. The at least one betacyanin, a red pigment, is preferably obtained from beet root (Beta vulgaris) or other similar herb which protects against skin cell damage from UV irradiation exposure, specifically UV-B radiation.

The at least one antioxidant component of the present invention preferably includes selenium. Selenium generally supports cellular health, has antioxidant functions, and supports the skin's ability to protect from photodamage alone or in combination with the Vitamin C, Vitamin E, and at least one carotenoid component. Matrix metalloproteinases (MMPs) are enzymes contained in the skin which support healthy skin immunity. Selenium modulates MMPs which are supportive of the skin's defenses against photodamage and reduces the activity of MMPs which break down collagen. In one preferred embodiment, selenium includes at least one of the following forms: selenate, selenite, selenomethionine, selenocysteine, yeast containing high amounts of selenium, selenium ascorbate, selenium glycinate, selenium aspartate, selenium citrate, selenium fumarate, selenium malate, or other similarly nutritional form of selenium.

The at least one antioxidant component of the present invention includes zinc. Zinc generally supports healthy skin immunity, and has antioxidant functions. Zinc is required by MMPs to support healthy skin immunity. In one preferred embodiment, zinc includes at least one of the following forms: zinc sulfate, zinc oxide, zinc gluconate, zinc aspartate, zinc citrate, zinc picolinate, zinc acetate, zinc carbonate, zinc fumate, zinc malate, zinc glycinate, zinc ascorbate, zinc alpha-keto-glutarate, or other similarly nutritional forms of zinc.

The at least one vitamin component of the present invention preferably includes Vitamin E, Vitamin C and Vitamin D or a pharmaceutically active source thereof; and para-aminobenzoic acid (PABA).

The at least one vitamin component of the present invention preferably includes Vitamin E, or a pharmaceutically active source of Vitamin E. Vitamin E increases the tolerance of unexposed skin to UV irradiation and supports healthy response to UV exposure especially when working in combination with beta-carotene or other similar carotenoid. Additionally, Vitamin E in combination with Vitamin C, or a pharmaceutically active source of Vitamin C, protects DNA found in dermal layer cells of the skin from mutation and photodamage. In one preferred embodiment, Vitamin E includes d-alpha tocopherol. In another embodiment, Vitamin E includes mixed tocopherols or tocotrienols or other similarly therapeutic ingredients.

The at least one vitamin component of the present invention preferably includes Vitamin C, or a pharmaceutically active source of Vitamin C. In one preferred embodiment, the Vitamin C includes ascorbic acid, ester c, or other similarly therapeutic Vitamin C esters. Vitamin C in combination with Vitamin E, or a pharmaceutically active source of Vitamin E, protects DNA found in dermal layer cells of the skin from mutation and photodamage. Vitamin C also promotes healthy skin and the production of collagen in the skin.

The at least one vitamin component of the present invention also preferably includes Vitamin D, or a pharmaceutically active source of Vitamin D. The use of sunscreen having an SPF greater than SPF 8 blocks the body's natural production of Vitamin D. Regular Vitamin D intake is necessary because Vitamin D helps to improve bone and muscle health. Generally, Vitamin D is derived from diet and from UV radiation exposure. If the body's production of Vitamin D is deficient then Vitamin D levels can most safely be increased through dietary supplements or fortified foods. The present invention supplements the body's Vitamin D intake since it blocks the body's natural production of Vitamin D by protecting the skin from UV irradiation.

The at least one vitamin component of the present invention also preferably includes para-aminobenzoic acid (PABA) or other similar nutrient. PABA acts as a sunscreen agent providing protection against UV irradiation when applied topically and supports healthy collagen and tissue function when taken orally.

The at least one immuno-modulatory agent of the present invention preferably includes aloe or other similar immuno-modulatory agent. Aloe polysaccharides are generally used to enhance the skin's immune function and management of inflammatory changes from UV irradiation and to provide anti-aging benefits for the skin. In another preferred embodiment, the at least one immuno-modulatory agent includes at least one of the following: Echinacea, arabinogalactan (Western larch extract), maitake, AHCC, achyranthan, astragalus, or other similar immuno-modulatory agent.

The at least one herbal component of the present invention preferably includes Withania somnifera. Withania somnifera is an herb which supports healthy immune function and protects against skin damage from UV irradiation.

In one preferred embodiment, the composition includes at least one anti-oxidant component, at least one vitamin component, at least one immuno-modulatory agent and at least one herbal component, the formulation including at least one or more of the following ingredients:

Beta-carotene;

Vitamin E;

Vitamin C;

Pycnogenol®;

Selenium;

Zinc;

Vitamin D;

Para-aminobenzoic acid;

Green Tea Extract including catechins and polyphenols;

Pomegranate Extract including ellagic acid;

Beet Root Extract including betacyanins;

Aloe polysaccharides; and

Withania somnifera.

The individual components may be included in the composition in any of their pharmaceutically active or acceptable salt forms.

In a further preferred embodiment, the composition includes at least one anti-oxidant component, at least one vitamin component, at least one immuno-modulatory agent, and at least one herbal component, the formulation including at least one or more of the following ingredients in amounts which represent a daily therapeutic administration of the composition as follows:

Beta-carotene from approximately 3 mg to 25 mg;

Vitamin E from approximately 200 IU to 500 IU;

Vitamin C from approximately 60 mg to 250 mg;

Pycnogenol® from approximately 20 mg to 120 mg;

Selenium from approximately 55 μg to 200 μg;

Zinc from approximately 10 mg to 30 mg;

Vitamin D from approximately 100 IU to 400 IU;

Para-aminobenzoic acid from approximately 50 mg to 200 mg;

Green tea extract including catechins and polyphenols up to 320 mg;

Beet root including betacyanins;

Pomegranate extract including ellagic acid from approximately 15 mg to 100 mg;

Aloe Polysaccharides from approximately 50 mg to 160 mg; and

Withania somnifera from approximately 50 mg to 250 mg.

In one preferred embodiment, the composition is suitable for oral administration, although any suitable route of administration may be used in a therapeutically effective dosage of the composition. Other routes of administration include, for example, parenteral, intravenous, topical or other like forms of administration. The preferable dosage form for oral administration includes but is not limited to tablets, capsules, gel caps, caplets or other suitable forms of oral administration. The preferable dosage form for other routes of administration include but are not limited to solutions, liquids, suppositories or other suitable forms for administration other than oral administration.

In one preferred embodiment, the method of administering the therapeutically effective dosage of the composition is oral and provides protection for the skin against the effects of UV irradiation that is not affected by skin type, exposure to water, and sweating.

In one preferred embodiment, the method of administering the therapeutically effective dosage of the composition is oral and the skin protection provided against the effects of UV irradiation is supplemented by the application of a topical sunscreen to provide adequate protection from UV irradiation and against all ranges of UV-B and UV-A irradiation.

The nutritional product, dietary supplement or pharmaceutical composition used in the present invention includes the active ingredients as described above and may contain other inactive ingredients, pharmaceutically active carriers, and excipients. The nutritional product, dietary supplement or pharmaceutical composition may optionally include other therapeutic ingredients.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

I claim:

1. A composition for use in a nutritional product, dietary supplement or pharmaceutical composition comprising:
at least one antioxidant component in an amount sufficient to protect the skin against damage caused by UV irradiation, to maintain normal skin immune response and to scavenge for free radicals resulting from UV irradiation;
at least one vitamin component in an amount sufficient to protect the skin from photodamage, to promote healthy skin and the production of collagen, and to supplement Vitamin D;
at least one immuno-modulatory agent in an amount sufficient to reduce skin inflammation resulting from UV irradiation; and
at least one herbal component in an amount sufficient to support healthy skin immune function and to protect against skin damage from UV irradiation.

2. The composition of claim 1 wherein the at least one antioxidant component comprises beta-carotene.

3. The composition of claim 1 wherein the at least one antioxidant component comprises a polyphenol-containing compound.

4. The composition of claim 3 wherein the polyphenol-containing compound comprises pine bark extract.

5. The composition of claim 3 wherein the polyphenol-containing compound comprises Pycnogenol®.

6. The composition of claim 3 wherein the polyphenol-containing compound comprises green tea extract.

7. The composition of claim 1 wherein the at least one antioxidant component comprises ellagic acid.

8. The composition of claim 1 wherein the at least one antioxidant component comprises a betacyanin.

9. The composition of claim 8 wherein the betacyanin comprises beet root extract.

10. The composition of claim 1 wherein the at least one antioxidant component comprises Selenium or any active salt thereof.

11. The composition of claim 1 wherein the at least one antioxidant component comprises Zinc or any active salt thereof.

12. The composition of claim 1 wherein the at least one vitamin component comprises Vitamin E or any pharmaceutically active source thereof.

13. The composition of claim 1 wherein the at least one vitamin component comprises Vitamin C or any pharmaceutically active source thereof.

14. The composition of claim 1 wherein the at least one vitamin component comprises Vitamin D or any pharmaceutically active source thereof.

15. The composition of claim 1 wherein the at least one vitamin component comprises PABA or any pharmaceutically active source thereof.

16. The composition of claim 1 wherein the at least one immuno-modulatory agent comprises aloe.

17. The composition of claim 1 wherein the at least one herbal component comprises Withania somnifera.

18. A method for the protection of skin against the effects of UV irradiation which comprises administering:
at least one antioxidant component in an amount sufficient to protect the skin against damage caused by UV irradiation, to maintain normal skin immune response and to scavenge for free radicals resulting from UV irradiation;
at least one vitamin component in an amount sufficient to protect the skin from photodamage, to promote healthy skin and the production of collagen, and to supplement Vitamin D;
at least one immuno-modulatory agent in an amount sufficient to reduce skin inflammation resulting from UV irradiation; and
at least one herbal component in an amount sufficient to support healthy skin immune function and to protect against skin damage from UV irradiation.

19. The method of claim 18 wherein administration comprises oral administration.

20. The method of claim 19 wherein the composition provides skin protection against the effects of UV irradiation and is administered in combination with at least one topically applied composition to supplement the skin protection against the effects of UV irradiation.
